(19) United States
(12) Patent Application Publication
(10) Pub. No.: US 2019/0029987 A1
(43) Pub. Date: Jan. 31, 2019

(54) DIMETHYL FUMARATE (DMF) FOR PREVENTION OR TREATMENT OF GOUT, ACNE, DIABETES, VITILIGO AND/OR PYODERMA GANGRENOSUM

(71) Applicant: UNIVERSITAT ZURICH, Zurich (CH)

(72) Inventors: Hans-Dietmar BEER, Zurich (CH); Martha GARSTKIEWICZ, Zurich (CH); Lars E. FRENCH, Zurich (CH)

(73) Assignee: UNIVERSITAT ZURICH, Zurich (CH)

(21) Appl. No.: 16/077,476

(22) PCT Filed: Feb. 10, 2017
(86) PCT No.: PCT/EP2017/053029

§ 371 Date: Aug. 12, 2018

(30) Foreign Application Priority Data
Feb. 12, 2016 (EP) 16155554.5
Jun. 21, 2016 (EP) 16175524.4
Jun. 30, 2016 (EP) 16177215.7
Dec. 6, 2016 (EP) 16202534.0

(51) Int. Cl. A61K 31/225; A61P 17/10; A61P 19/06; A61P 3/10

(52) U.S. Cl. CPC A61K 31/225; A61P 3/10; A61P 19/06; A61P 17/10

(57) ABSTRACT

A compound specified by formula (I), in particular dimethylfumarate (trans-1,2-ethylenedicarboxylic acid dimethyl ester) is provided for use in prevention or therapy of gout, acne, pyoderma gangrenosum, Vitiligo, cardiovascular disease, metabolic syndrome, diabetes and/or complications of diabetes. Also provided are a dosage form comprising said compound and a method of treatment comprising administration of said compound to a patient in need thereof.

### BACKGROUND OF INVENTION

Dimethyl fumarate (DMF, CAS number 624-49-7) is approved as a drug for the treatment of psoriasis and multiple sclerosis (MS).

DMF is known to be an NRF2 (Nuclear factor erythroid 2 related factor 2) activator: it activates the basic leucine zipper transcription factor NRF2. The expression of NRF2 target genes has thus been generally assumed to underlie the therapeutic effect of DMF.

Other known NRF2 activators include Sulforaphane (SFN), tertiary butylhydrochinone (tBHQ), CDDO-imidazolide and 15-deoxy-Δ-12,14-prostaglandin J₂ (15d-PGD₂). Several contradictory and ambiguous reports exist however with regard to the mode of action of NRF2 activators and the underlying molecular mechanism. The controversial issues can be summarized as follows:

NRF2 and its target genes are involved in cytoprotection from xenobiotic and oxidative stress. Their function has thus mainly been regarded as cell protective and anti-apoptotic. More recently, NRF2 has been discovered to be involved in regulation of inflammasome-related processes. Inflammasomes are multiprotein complexes that activate the protease caspase-1, which in turn activates the proinflammatory cytokines pro-interleukin (prolL)-1β and -18. Inflammasomes thus play a crucial role in both acute and chronic inflammation and in conditions caused by inflammatory processes.

NRF2 loss of function (knock-out, knock down) prevents inflammasome activation, indicating that expression of NRF2 target genes is required to activate the inflammasome. On the other hand, NRF2 activators also prevent inflammasome activation (this was not shown for DMF), indicating that NRF2 target genes might be involved in inhibiting the inflammasome.

NRF2 activators induce stabilisation of NRF2 and translocation to the nucleus, which in turn induces expression of NRF2 target genes. It was recently reported however, that inhibition of the inflammasome by the NRF2 activator Sulforaphane is independent of NRF2 and its target genes. If the effect of Sulforaphane is independent of NRF2, it cannot reasonably be expected that other NRF2 activators would also have a beneficial effect in inflammasome-related diseases.

Importantly, recent results also suggest that the effect of DMF in the treatment of MS is independent of NRF2 and depends on hydroxycarboxylic acid receptor 2 instead.

These examples illustrate that the mode of action of NRF2 and NRF2 activators in inflammasome-related processes is highly debated and far from understood. Thus, the skilled person would not be led to believe that administration of NRF2 activators is necessarily beneficial in inflammasome-related diseases and would thus not use DMF for treatment of such diseases.

Currently, interleukin-1 (IL-1) blockers are used for the treatment of inflammasome-related diseases. Alternative and complementary medicaments would be highly desirable.

The problem underlying the present invention is to provide a means for treating conditions that are caused by activation of the inflammasome, in particular gout, acne and diabetes, more particularly acne vulgaris and type 2 diabetes. This problem is solved by the subject matter of the independent claims.

### DESCRIPTION OF THE INVENTION

According to a first aspect of the invention, a compound specified by formula (I)

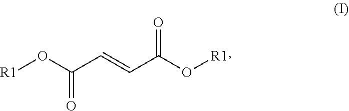

wherein each R1 is independently selected from H and C₁-C₆ alkyl, is provided for use in prevention or therapy of gout, acne, pyoderma gangrenosum, Vitiligo, cardiovascular disease, metabolic syndrome, diabetes and/or complications of diabetes.

In all of the abovementioned conditions, inflammasome activation is an important pathophysiological mechanism. The skilled person is aware that a patient suffering from one of the abovementioned conditions would benefit from a treatment that allows to inhibit the inflammasome and to control inflammasome activity.

Within the context of the present specification, the term “cardiovascular disease” has its general meaning known in the art and is used to classify conditions that affect the heart, heart valves, blood, and vasculature of the body. Cardiovascular diseases include endothelial dysfunction, coronary artery disease (CAD), angina pectoris, myocardial infarction, acute coronary syndrome (ACS), atherosclerosis, congestive heart failure, hypertension, cerebrovascular disease, stroke, transient ischemic attacks, deep vein thrombosis, peripheral artery disease, cardiomyopathy, arrhythmias, aortic stenosis, and aneurysm. Such diseases frequently involve atherosclerosis.

Within the context of the present specification, the term “metabolic syndrome” has its general meaning known in the art. It is a clustering of at least three of the five following medical conditions: abdominal (central) obesity, elevated blood pressure, elevated fasting plasma glucose, high serum triglycerides, and low high-density lipoprotein (HDL) levels. Metabolic syndrome is associated with the risk of developing cardiovascular diseases and diabetes.

Within the context of the present specification, the term “complications of diabetes” has its general meaning known in the art. Acute complications of diabetes include diabetic ketoacidosis, nonketonic hyperosmolar coma, hypoglycemia, diabetic coma, respiratory infections and periodontal disease. Possible chronic complications of diabetes include microangiopathy, diabetic cardiomyopathy, diabetic neuropathy, diabetic nephropathy, diabetic retinopathy, diabetic encephalopathy (including, but not limited to, Alzheimer's type dementia), macrovascular disease, cardiovascular disease, diabetic foot (foot complications due to nerve damage in the feet or poor blood flow to the feet) and skin infections. Complications are far less common and less severe in people who have well-controlled blood sugar levels.

In certain embodiments, the compound is provided for use in prevention or therapy of acne vulgaris.

In certain embodiments, the compound is provided for use in prevention or therapy of type 2 diabetes. In certain embodiments, R1 is a methyl, ethyl, propyl or butyl.

In certain embodiments, said compound is dimethylfumarate (trans-1,2-ethylenedicarboxylic acid dimethyl ester; CAS No, 624-49-7).

In certain embodiments, the compound is ethylhydrogenfumarate (with one R1 being ethyl and the other one being H), or a salt of ethylhydrogenfumarate. In certain embodiments, the compound is a magnesium salt of ethylhydrogenfumarate. In certain embodiments, the compound is a calcium salt of ethylhydrogenfumarate. In certain embodiments, the compound is a zinc salt of ethylhydrogenfumarate.

In certain embodiments, the active ingredient employed in prevention or therapy of gout, acne, pyoderma gangrenosum, Vitiligo, cardiovascular disease, metabolic syndrome, diabetes and/or complications of diabetes, is a mixture of dimethylfumarate and magnesium, calcium and zinc salts of ethylhydrogenfumarate. A commercial preparation marketed as fumaderm comprises, per administration form, 30 mg dimethylfumarate, 67 mg ethylhydrogenfumarate calcium salt, 5 mg ethylhydrogenfumarate magnesium salt and 3 mg zinc salt. Also commercially available is a lozenge comprising 120 mg dimethylfumarate and 95 mg ethylhydrogenfumarate, the latter being administrated as the respective salts of calcium (87 mg), magnesium (5 mg) and zinc (3 mg). These administration forms are similarly considered as possible embodiments of the invention.

The inventors demonstrate that the compound is effective in low doses, thus minimizing potential side effects. DMF is being used as a medicament for decades and has been shown to be well tolerated. Alternative compounds capable of inhibiting the inflammasome, like SFN (GAS No 4478-93-7) or 15d-PGJ₂, (GAS 87893-55-8) also affect other cellular pathways and their use as a medicament is thus likely to be less safe.

According to a second aspect of the invention, a dosage form comprising the compound according to the first aspect of the invention is provided for use in prevention or therapy of gout, acne, pyoderma gangrenosum, Vitiligo, cardiovascular disease, metabolic syndrome, diabetes and/or complications of diabetes.

In certain embodiments, the dosage form comprises the compound as specified according to the first aspect of the invention alone or together with one or more pharmaceutically acceptable excipient or carrier.

In certain embodiments, the dosage form is a peroral formulation, particularly a tablet, capsule, lozenge, powder, solution or syrup.

In certain embodiments, the dosage form is a topical medication, particularly an epicutaneous medication, more particularly a cream, gel, ointment or lotion.

In certain embodiments, the dosage form is formulated as a cream. In certain embodiments, the dosage form is formulated as a lotion. In certain embodiments, the dosage form is formulated as a ointment. In certain embodiments, the dosage form is formulated as a spray.

The skilled artisan is aware of a broad range of possible recipes for providing topical formulations, as exemplified by the content of Benson and Watkinson (Eds.), Topical and Transdermal Drug Delivery: Principles and Practice (1st Edition, Wiley 2011, ISBN-13: 978-0470450291); and Guy and Handcraft: Transderrnal Drug Delivery Systems: Revised and Expanded (2^{nd} Ed., CRC Press 2002, ISBN-13: 978-0824708610); Osborne and Arnann (Eds.): Topical Drug Delivery Formulations (1^{3t} Ed. CRC Press 1989; ISBN-13: 978-0824781835).

The dosage form may be administered alone or in combination with one or more therapeutic agents, particularly in combination with an interleukin-1 inhibitor.

According to an alternative aspect of the invention, a method of treatment or prevention of gout, acne, pyoderma gangrenosum, Vitiligo, cardiovascular disease, metabolic syndrome, diabetes and/or complications of diabetes is provided, comprising administration of the compound according to the first aspect of the invention to a patient in need thereof. Administration may be effected by any of the aforementioned means.

The compound may be given to a patient already diagnosed with gout, acne, pyoderma gangrenosum, Vitiligo, cardiovascular disease, metabolic syndrome, diabetes and/or complications of diabetes, or to a patient being suspected of suffering from gout, acne, pyoderma gangrenosum, Vitiligo, cardiovascular disease, metabolic syndrome, diabetes and/or complications of diabetes. Alternatively, the compound may be used as a prophylactic for patients that are at risk of developing gout, acne, pyoderma gangrenosum, Vitiligo, cardiovascular disease, metabolic syndrome, diabetes and/or complications of diabetes.

The invention is further illustrated by the following items, examples and figures, from which further embodiments and advantages can be drawn. These examples are meant to illustrate the invention but not to limit its scope.

Items

1. A compound specified by formula (I)

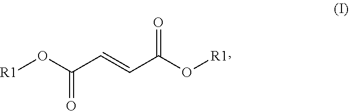

wherein each R1 is independently selected from H and C₁-C₆ alkyl, for use in prevention or therapy of gout, acne, pyoderma gangrenosum, Vitiligo, cardiovascular disease, metabolic syndrome, diabetes and/or complications of diabetes.

2. The compound specified by formula (I), wherein each R1 is methyl (CH₃), for use in prevention or therapy of gout, acne, pyoderma gangrenosum, Vitiligo, cardiovascular disease, metabolic syndrome, diabetes and/or complications of diabetes.

3. The compound specified by formula (I), wherein one R1 is ethyl (CH₂CH₃) and the other R1 is selected from H and C₁-C₆ alkyl, for use in prevention or therapy of gout, acne, pyoderma gangrenosum, Vitiligo, cardiovascular disease, metabolic syndrome, diabetes and/or complications of diabetes.

4. The compound according to item 3, wherein the other R1 is H, for use in prevention or therapy of gout, acne, pyoderma gangrenosum, Vitiligo, cardiovascular disease, metabolic syndrome, diabetes and/or complications of diabetes.

A preparation comprising diethylfumarate and one or several salts of ethylhydrogenfumarate, particularly salts selected from the magnesium salt, calcium salt and zinc salt of ethylhydrogenfumarate.

6. A dosage form comprising the compound according to any one of items 1 to 5 for use in prevention or therapy of gout, acne, pyoderma gangrenosum, Vitiligo, cardiovascular disease, metabolic syndrome, diabetes and/or complications of diabetes.

7. A dosage form comprising the compound according to item 2 and the compound according to item 3 for use in prevention or therapy of gout, acne, pyoderma gangrenosum, Vitiligo, cardiovascular disease, metabolic syndrome, diabetes and/or complications of diabetes.

8. A method of treatment or prevention of gout, acne, pyoderma gangrenosum, Vitiligo, cardiovascular disease, metabolic syndrome, diabetes and/or complications of diabetes, comprising administration of the compound, preparation or dosage form according to any one of items 1 to 7 to a patient in need thereof.

### EXAMPLES

Abbreviations: DMF: dimethyl fumarate, Nrf2: nuclear factor erythroid derived 2, like 2, ROS: reactive oxygen species, KEAP1: Kelch-like ECH-associated protein 1, Cul3: Cullin 3, Rbx1: RING-box protein 1, SFN: sulforaphane, MS: multiple sclerosis, NLRP3: NACHT, LRR and PYD domains-containing protein 3, ASC: apoptosis-associated speck-like protein containing a CARD, IL: interleukin, BMDCs: bone marrow-derived dendritic cells, HPKs: human primary keratinocytes, tBHQ: tert-butylhydroquinone, 15d-PGJ2: 15-deoxy-D-prostaglandin J2, ca: constitutively active, PBMCs: peripheral blood mononuclear cells, MSU: monosodium urate, co-IP: co-immunoprecipitation.

Nrf2 Expression is Required for Efficient Inflammasome Activation

To determine if the basal activity of Nrf2 is required for inflammasome activation, the inventors generated bone marrow-derived dendritic cells (BMDCs) from Nrf2-deficient mice and wildtype littermates. The inventors primed the cells with LPS, activated the NLRP3 as well as the AIM2 inflammasomes by several potent inducers, and analysed the secretion of mature IL-1β as a readout for caspase-1 activation. Secretion of IL-1β by Nrf2-deficient BMDCs was severely impaired as demonstrated by Western blot and ELISA (FIG. 6 A, B). As a control, the inventors analysed expression of pro-IL-1β and of several inflammasome proteins at the mRNA and protein level. However, expression of these genes was not significantly affected by the loss of Nrf2 (FIG. 6C,D).

Human primary keratinocytes (HPKs) constitutively express pro-IL-1β and inflammasome proteins. Therefore, priming is not required for secretion of mature IL-1β and -18 mediated by UVB irradiation-induced inflammasome activation, Treatment of HPKs with the Nrf2 activating compounds SFN, DMF, tert-butylhydroquinone (tBHQ) or 15-deoxy-D-12,14116-prostaglandin J2 (15d-PGJ2) resulted in fast and robust stabilisation and nuclear accumulation of Nrf2 protein, whereas expression of the other Nrf2 complex proteins Keap1, Cul3, and Rbx1 was not affected (FIG. 6E,F). Nrf2 stabilisation and nuclear accumulation was accompanied by induction of classical Nrf2 target genes (FIG. 6G). In addition, knock-down of Keap1 or Cul3 expression induced stabilisation of Nrf2, its nuclear accumulation, and enhanced target gene expression (FIG. 7A-C), These experiments demonstrate that the Nrf2 pathway is functional in HPKs.

Therefore, the inventors knocked down Nrf2 expression in HPKs using siRNA and analysed inflamrnasorne activation upon UVB irradiation (FIG. 1A-C), In Nrf2 knock-down HPKs caspase-1 activation was inhibited, and secretion of IL-1β and -18 was reduced, demonstrating that Nrf2 expression is also required for efficient inflammasome activation in human keratinocytes.

Nrf2-Induced Gene Expression is not Involved in Inflammasome Regulation

Since Nrf2 is a transcription factor, it is likely that a reduction of Nrf2 target gene expression underlies the inhibition of the NLRP3 inflammasome upon ablation of Nrf2 expression. To determine if activation of Nrf2-mediated gene expression has the opposite effect and results in enhanced maturation of pro-IL-1p, the inventors characterised peritoneal macrophages isolated from transgenic mice expressing a constitutively active (ca) mutant of Nrf2 in myeloid cells. This mutant lacks the domain, which mediates binding to Keap1. However, secretion of mature IL-1β and consequently NLRP3 inflammasome activation was not changed upon caNrf2 expression (FIG. 1D,E), although expression of Nrf2 target genes was induced (FIG. 1F).

To further address the possibility that Nrf2 target genes regulate NLRP3 inflammasome activation, the inventors performed experiments in HPKs. The inventors transduced these cells with lentiviral constructs encoding wild-type Nrf2 or Keap1, or mutant proteins. After induction of expression the cells were irradiated with UVB, resulting in inflammasome activation as reflected by secretion of mature IL-1β (FIG. 1G). As a control, mRNA levels of Nrf2 target genes were determined (FIG. 1H), Overexpression of wild-type Nrf2 indeed increased secretion of IL-1p (FIG. 1G). However, and consistent with the results obtained with macrophages from caNrf2-transgenic mice, overexpression of the caNrf2 mutant increased target gene expression to a similar extent, but did not enhance pro-IL-1p maturation. An Nrf2 mutant lacking the nuclear localization sequence (Nrf2_NLS) slightly increased target gene expression, but strongly increased IL-1β production (FIG. 1G,H). Wild-type Keap1 as well as a mutant, which cannot mediate Nrf2 degradation, increased IL-1β in the supernatant of HPKs, although the proteins influenced Nrf2 target gene expression in an opposite manner. These results demonstrate that NLRP3 inflammasome activation is not correlated with the expression of Nrf2 target genes.

Nrf2 Activators Inhibit NLRP3 Inflammasome Activation

To determine the effects of Nrf2 activating compounds on inflammasome activation, the inventors treated keratinocytes with different doses of SFN, tBHQ, DMF or 15d-PGJ2 and irradiated the cells with UVB. These compounds inhibited inflammasome activation in a dose-dependent manner as reflected by detection of reduced amounts of processed caspase-1 and mature IL-1β and -18 in the supernatant (FIG. 2A, FIG. 8A-C). SFN and 15d-PGJ2 were much more efficient than tBHQ and DMF, The anti-inflammatory effect of Nrf2 activators is not restricted to human keratinocytes, since they also inhibited IL-1β secretion in the human monocytic cell line THP-1 (FIG. 2B) and in human peripheral blood mononuclear cells (PBMCs) (FIG. 2C). Nrt2 activating compounds strongly inhibited pyroptosis in inflammasome activated THP-1 cells, reflected by the reduced release of the cytoplasmic enzyme lactate dehydrogenase (LDH) (FIG. 8D). However, since mature IL-1β did not accumulate in these cells, the experiment demonstrates that SFN and 15-PGJ2 indeed inhibit inflammasome activation rather than only pyroptosis. As the Nrf2 activators were added to the cells only 15 to 30 min prior to inflammasome activation, it is unlikely that Nrf2 target genes are involved in inflammasome inhibition. To further test this possibility, the inventors treated HPKs (FIG. 2D) or THP-1 cells (FIG. 2E) with cycloheximide, which blocks protein synthesis (FIG. 7E). If added just before treatment of cells with SFN, cycloheximide did not prevent inflammasome inhibition by the Nrf2 activator. These experiments provide strong evidence that induction of Nrf2 target genes does not underlie inflammasome inhibition by SFN.

DMF Dampens Inflammasome-Dependent Inflammation

An important open question is whether Nrf2 activating compounds are able to block inflammasome-dependent inflammation in vivo. Although DMF is used as a drug for the treatment of the inflammatory diseases psoriasis and MS, its mode of action is poorly characterised. However, in both diseases an involvement of inflammasomes is discussed. Monosodium urate (MSU) crystal-induced peritonitis is a mouse model of inflammation and gout, which is dependent on IL-1, IL-1R1, MyD88 and the NLRP3 inflammasome. Recently, it has been shown that Nrf2 expression is required for this type of inflammation. Most importantly, high concentrations of the Nrf2 activators 15d-PGJ2 and SFN, when injected into the peritoneum, blocked inflammasome activation and reduced MSU-induced peritonitis. The inventors chose a different way of administration and supplied mice with SFN or DMF by oral gavage to determine a potential anti-inflammatory activity of the Nrf2 activators in vivo (FIG. 3). Since DMF was less potent in inflammasome inhibition than SFN at the same concentrations (FIG. 8 A, B), the inventors treated mice with DMF for six instead of two days for SFN before induction of peritonitis (FIG. 3 A, D). The inventors analysed the cellular infiltrate in the peritoneum 6 h post injection of MSU crystals. The number of neutrophils was significantly reduced in SFN- and DMF-treated compared to control mice (FIG. 3 B, E). As a control for the SFN and DMF treatment the inventors determined expression of Nrf2 target genes in the liver and found increased mRNA expression (FIG. 3 C, F). These results demonstrate that SFN and DMF, when orally administered, inhibit inflammation in an NLRP3 inflammasome-dependent mouse model.

NLRP3 Inflammasome Activation Downregulates Nrf2 Expression

Next, the inventors investigated the activity of Nrf2 upon activation of the NLRP3 inflammasome, since inflammasome activation is ROS-dependent. Interestingly, UVB irradiation of HPKs induced a strong and fast downregulation of Nrf2 protein levels, followed by reduction of Nrf2 target gene expression, while caspase-1 activity was induced (FIG. 4A,C). This is surprising, since UVB irradiation is a strong inducer of ROS production and it can be anticipated that the cells would benefit from Nrf2 activation. UVB-induced secretion by HPKs requires expression of NLRP1 and NLRP3 [21]. Both nigericin and MSU crystals are considered as “true” NLRP3 activators, but HPKs cannot phagocytose MSU crystals. Therefore, the inventors treated HPKs with nigericin only and THP-1 cells with either of these NLRP3 activators. These treatments also resulted in a fast downregulation of Nrf2 protein levels (FIG. 4B,D) and target gene expression (FIG. 4E), while only UVB irradiation strongly downregulated Nrt2 mRNA expression (FIG. 4C,E). Therefore, NLRP3 inflammasome activation most likely induces Nrf2 protein degradation. Interestingly, this effect does not require caspase-1 expression and activity and is partially independent of Keap1 as determined by siRNA-mediated knock-down of these proteins in HPKs or knockouts in THP-1 cells (FIG. 4F,G). However, inflammasome activation-induced Nrf2 degradation was blocked upon ablation of ASC or NLRP3 expression (FIG. 4G) and upon treatment of cells with the ROS blocker PDTC or the Ca2+ chelator BAPTA-AM (Supplementary FIG. 2D). Keap1 ablation resulted in reduced pro-IL-1β and NLRP3 levels (FIG. 4G), which might be explained by impaired TLR4 signalling or inhibition of pro-IL-1β expression by Nrf2. However, inflammasome function was not impaired, as reflected by normal processing of pro-IL-18 (FIG. 4H). Although Keap1 expression is partially dispensable for Nrf2 degradation by inflammasome activation, the transcription factor is directed to the proteasome under these conditions, since Nrf2 degradation was inhibited by the proteasome inhibitor MG132 (FIG. 4I and FIG. 7D).

The Nrf2/Keap1/Cul3/Rbx1 Complex Physically Interacts with Caspase-1

The inventors' experiments demonstrate that Nrf2 target genes are most likely not involved in the cross-talk between Nrf2 and the NLRP3 inflammasome, pointing to a novel mechanism, by which the transcription factor is linked to inflammation. Since overexpression experiments in HPKs (FIG. 1G,H) suggested a correlation between the amount of cytoplasmic Nrf2/Keap1 and inflammasome activation, it seems possible that Nrf2 supports NLRP3 inflammasome activation by a direct or indirect physical interaction with the immune complex. To address this point, the inventors performed co-immunoprecipitation (co-IP) experiments with an antibody for caspase-1 and lysates of HPKs. However, the inventors were not able to detect an interaction between caspase-1 and Nrf2 (results not shown). Interestingly, however, interaction of caspase-1 with Rbx1 was found. The specificity of the band was verified by knock-down of Rbx1 expression (FIG. 5A). In addition, the inventors overexpressed a FLAG-tagged version of caspase-1 in HPKs and precipitated the protease with an ANTI-FLAG M2 Affinity Gel (FIG. 5B). In this precipitate endogenous Nrf2, Keap1, Cul3, and Rbx1 were detected, demonstrating that overexpressed caspase-1 interacts with these proteins. However, treatment of HPKs with SFN did not prevent the interaction between caspase-1 and Rbx1 (FIG. 5C). To address the question whether Nrf2 complex proteins interact with inflammasome proteins directly, the inventors performed co-IP experiments with lysates of transfected COS-1 or HEK293T cells. However, interactions of Nrf2, Keap1 and Rbx1 with caspase-1, pro-IL-1β and NLRP3 could not be detected in a reproducible manner (results not shown). These experiments demonstrate a physical crosstalk between the Nrf2 and NLRP3 complexes, which may explain the requirement of Nrf2 expression for NLRP3 inflammasome activation.

Most likely, this interaction is not direct, but mediated by unknown proteins. The fact that Rbx1 is bound to caspase-1 also upon SFN treatment of HPKs raises the possibility that Nrf2 activators inhibit inflammasome activation through a different molecular mechanism. To address this possibility, the inventors treated BMDCs from wild-type and Nrf2 knockout mice with SFN or vehicle (FIG. 5 D, E). Whereas Nrf2 ablation reduced IL-1β maturation and, therefore, inflammasome activation, SFN completely abolished secretion of the cytokine independently of Nrf2 expression. Most importantly, inflammasome inhibition by SFN is Keap1 independent (FIG. 4H). These experiments demonstrate that Nrf2 ablation and SFN inhibit NLRP3 inflammasome activation by different molecular mechanisms. In order to analyse, at which level SFN blocks inflammasome activation, the inventors determined the formation of ASC specks in SFN-treated and inflammasome activated THP1 cells (FIG. 5F). Formation of ASC specks is an upstream event of inflammasome activation. Interestingly, SNF treatment prevented oligomerization of the adaptor protein demonstrating that inflammasome inhibition by the compound is an upstream effect.

Materials and Methods

Materials

SFN, DMF, 15d-PGJ2, tBHQ, zymosan, ATP, poly(dA:dT), cycloheximide, puromycin, and doxycycline were purchased from Sigma (Munich, Germany), nigericin from Enzo Life Sciences (New York, US-NY), MG132 from Calbiochem (Darmstadt, Germany), and blasticidin from Invivogen (Toulouse, France). Release of IL-1p was determined by ELISA according the instructions of the manufacturer (R&D Systems, Minneapolis, US-MN). MSU crystals were prepared by crystallisation of a supersaturated solution of uric acid under mildly basic conditions. Briefly, uric acid was added to a solution of NaOH, the solution was boiled until the uric acid was dissolved and passed through a filter. NaCl was added and crystallisation was performed at 4° C. Crystals were filtered, then dried using a speedvac, weighted and autoclaved.

Constructs

Expression constructs for Nrf2, dnNrf2 (Alam et al., J Biol Chem 1999. 274: 26071-26078), caNrf2 (Schafer et al., Genes Dev 2010. 24: 1045-1058), and Keap1 were kindly provided by Prof. Werner. Lentiviral system and vectors were described by (Campeau et al., PLoS One 2009. 4: e6529). pLenti CMVtight Puro DEST (w768-1) (Addgene: 26430), pLenti CMV rtTA3 Blast (w756-1) (Addgene: 26429), pENTR1A no ccDB (w48-1) (Addgene: 17398), pLenti CMVtight eGFP Puro (w771-1) (Addgene: 26431).

Antibodies

Murine: IL-1β (R&D systems, AF-401-NA), caspase-1 (Santa Cruz, Santa Cruz, US-CA; sc-514), Asc (Adipogen, Liestal, Switzerland; AL177), β-actin (Sigma, AC-15). Human: Nrf2 (Santa Cruz, sc-13032), caspase-1 (Santa Cruz, sc-622), Keap1 (Santa Cruz, sc-15246), Rbx1 (Abeam, Cambridge, UK; ab133565), β-actin (Sigma, AC-15), IL-1β (R&D systems, MAB 201), IL-18 (MBL, Woburn, US-MA; PM014), lamin A/C (Santa Cruz, se-6215), α-tubulin (Calbiochem, CP06), FLAG (M2, Sigma, F1804).

siRNAs

siRNAs were purchased from Microsynth (Balgach, Switzerland) or Sigma (Munich, Germany).

In Vivo Peritonitis Model

Mice were challenged with 2 mg of MSU crystals for 6 hours as previously described (Chen et a Olin Invest 2006. 116: 2262-2271).

Statistical Analysis

Statistical analysis was performed using the Prism Software (GraphPad Software; San Diego; CA, USA).

Mice

All animal experiments were approved by the local veterinary authorities (Zurich, Switzerland). Mice were kept in a pathogen-free animal facility according to the federal guidelines. Nrf2 knockout mice (Chan et al., Proc Natl Acad Sci USA 1996. 93: 13943-13948) were kindly provided by Dr. Yuet-Wai Kan, University of California, San Francisco. Mice expressing ca Nrf2 in myeloid cells were generated by mating of transgenic mice expressing Cre under control of the LysM gene promoter (Clausen et al., Transgenic Res 1999. 8: 265-277) with transgenic mice expressing caNrf2 under control of a β-actin promoter and CMV enhancer. To avoid expression of the transgene in all cells, the caNrf2 cDNA is flanked by loxP site, allowing expression of the caNrf2 transgene in the presence of Cre recombinase (Schafer et al., EMB© Mol Med 2012. 4: 364-379).

Cells

Human primary keratinocytes (HPKs) were isolated and propagated as described (Feldmayer et al., Curr Biol 2007. 17: 1140-1145). Briefly, HPKs were cultured in keratinocyte serum free medium (Gibco BRL, Paisley, Scotland), supplemented with epidermal growth factor (EGF) and bovine pituitary extract. For all experiments HPKs were used in passage 3. For transfection of specific siRNAs (Supplementary Table 1) HPKs were seeded at a density of 0.3-0.5×10° per 12 well. The day after, HPKs were transfected with 10 nM siRNA and 1 μl INTERFERin (Polyplus, Illkirch, France). If necessary, transfection was repeated 2 days later.

CRISPR/Cas9-Mediated Genome Editing in THP-1 Cells

gRNAs were designed using the Benchling online tool (https://benchling.com). Single stranded forward and reverse DNA oligos were ordered from Microsynth (Balgach, Switzerland). After phosphorylation and annealing of the oligos, they were ligated into the LentiCRISPR v2 vector (Addgene Plasmid #52961) described in (Sanjana et al., Nat Methods 2014. 11: 783-784) Lentivirus production as described above. THP-1 cells were transduced and 24 h later medium was changed. After additional 24 h, puromycin was added to a final concentration of 5 μg/ml for selection.

Quantitative Real-Time PCR (qRT-PCR)

qRT-PCR was performed with the LightCycler 480 SYBR Green Master or the FastStart Universal SYBR Green Master (both Roche, Rotkreuz, Switzerland) using total cellular RNA. Specific primer pairs (Supplementary Table 2) were designed to generate an approximately 150 bp fragment flanking an intron-exon border of the corresponding gene. The LightCycler 480 96-well version (ROCHE, Rotkreuz, Switzerland) or the ViiA 7 Real-Time FOR System (Life Technologies, Carlsbad, US-CA) was used for reaction and detection according the instructions of the manufacturer.

Lactate Dehydrogenase Assay (LDH)

Supernatants were harvested and after centrifugation (400×g) used for analysis. Cells were lysed in culture medium with 2% Triton X-100 for 10 min, LDH activity was determined according to the manufacturer (Cytotox 96 nonradioactive cytotoxicity assay, Promega, Madison, US-WC).

Co-Immunoprecipitation

Co-immunoprecipitation (co-IP) was performed with lysates of HPKs as described (Sollberger et al., J Immunol 2012. 188: 1992-2000). Briefly, HPKs were grown in 10 cm dishes, transfected with siRNA (scr or siRNA targeting Rbx1), and propagated to 80% confluency (3 days), Four dishes were harvested in 150 μl co-IP buffer with complete proteinase inhibitor (Roche, Rotkreuz, Switzerland), respectively. After treatment in a douncer, the lysates were centrifuged (20 min, 17 000×g). The supernatant was diluted 1: 1 with co-IP buffer and incubated with 20 μg antibody (caspase-1 or HA). After centrifugation, 150 μl (50 mg/ml) protein A Sepharose (GE Healthcare, Little Chalfront, UK) was added. After 90 min, the beads were washed four times with co-IP buffer and resuspended in 100 μl 2×loading SDS buffer. Alternatively, co-immunoprecipitation was performed using an ANTI-FLAG® M2 Affinity Gel (Sigma). HPKs were grown in 10 cm dishes and transduced with lentiviral constructs encoding FLAG-tagged caspase-1 or GFP as described above. After antibiotic selection for 3 days, expression of the gene of interest was induced by the addition of doxycycline. After 24 h, cells were collected in lysis buffer, centrifuged and resulting supernatants were subjected to immunoprecipitation following the manufacturer's protocol.

Analysis of ASC Oligornerization

ASC specks were analysed as described (Nara et al., Nat Immunol 2013. 14: 1247-1255),

Lentivirus Production and Transduction of HPKs

To generate keratinocytes, which overexpress a gene of interest in an inducible manner, the inventors used alentiviral system (Campeau et al., PLoS One 2009, 4: e6529), DNA was cloned from expression vectors into the pENTR1A no ccDB (w48-1) vector and subsequently subcloned into the lentiviral pLenti CMVtight Puro DEST (w768-1) vector. Lentivirus was produced by transfection of HEK 293T cells with a mix of either the pLenti CMVtight Puro DEST (w768-1) vector encoding the desired gene of interest or pLenti CMV rtTA3 Blast (w756-1) encoding a reverse tetracycline-controlled transactivator 3 (rtTA3) and the two packaging vectors psPAX2 and pMD2.G. 48 h post transfection, the supernatant of the HEK 293T cells was collected and centrifuged at 16′000×g for 4 h. The resulting virus pellet was resuspended in K-SFM and added to freshly thawed HPKs. 24 h after transduction, medium was changed and the cells were left for another 24 h before applying selection (blasticidin 1 μg/ml and puromycin 0.5 μg/ml), After 24-48 h, transduced HPKs were seeded in 12-well plates. Expression of the gene of interest was induced by adding doxycycline (1 μg/ml) for 20 h the day after.

### BRIEF DESCRIPTION OF THE FIGURES

FIG. 1 shows that Nrf2 expression is required for full inflammasome activation, but Nrf2 target genes are not involved in NLRP3 inflammasome regulation. (A-C) Human primary keratinocytes (HPKs) were transfected with specific siRNAs as indicated (scr; scrambled, VEGF: vascular endothelial growth factor (additional control), c1: caspase-1, N2: Nrf2), 3 d later (A, B) irradiated with UVB or (C) mock treated and harvested after 5 h. Inflammasome activation was analysed by (A) ELISA measurement of IL-1β in the supernatant or by (B) western blotting as indicated. Specific bands are marked with an asterisk. (C) Western blot for analysis of expression of Nrf2/Keap1 complex proteins and caspase-1 of mock-treated HPKs after transfection with caspase1, Nrf2 or control siRNAs, (D-F) Peritoneal macrophages were isolated from mice, which overexpress a constitutively active (ca) mutant of Nrf2 in myeloid cells and from control mice. Cells were treated as described (Supplementary FIG. 1A) and analysed for NLRP3 inflammasome activation by IL-1β measurement in supernatants by (D) ELISA or (E) Western blot. (F) Expression of the Nrf2 target genes sulfiredoxin 1 (Srxn1), glutamate-cysteine ligase, modifier subunit (Gclrn), and glutathione S-transferase P1 (Gstp1) was determined by qRT-PCR. (G, H) HPKs were transduced with lentiviral constructs encoding the indicated proteins (GFP: green fluorescent protein; dnNrf2: dominant negative Nrf2, not interacting with Keap1, no transcriptional activation domain; caNrf2: constitutively active Nrf2, no Keap1-binding domain; Nrf2_NLS: Nrf2 lacking nuclear localization domain; nt: not transduced). Transduced cells were selected by cultivation in antibiotic-containing medium for 1 d. Expression was induced with doxycycline 3 d later. (G) Cells were irradiated with UVB and 5 h later lysates and supernatants were harvested and analysed for the expression and activation of the indicated proteins by Western blot. For Nrf2, two different antibodies, targeting different epitopes were used. (H) HPKs were harvested, and expression of the indicated Nrf2 target genes was determined by qRT-PCR. (A-H) Representative experiments performed at least three times are shown. Statistics: (A) Error bars represent the mean±SD of a representative experiment performed in triplicates. One-way ANOVA was performed. (D) Error bars represent the mean±SD of a representative experiment performed with three mice per genotype. Mann-Whitney test was performed. ***P≤0.001,

FIG. 2 shows that Nrf2 activation blocks inflammasome activation. (A) HPKs were treated with the indicated concentrations of the Nrf2 activating compound tBHQ irradiated with UVB 30 min later and harvested after 5 h. ELISA measurements were performed for quantification of IL-1β secretion and Western blots for analysis of expression and activation of the indicated proteins. Specific bands are marked with an asterisk. (B) THP-1 cells were differentiated with PMA (27 nM) for 3 d, primed with upLPS (100 ng/ml) overnight, and 1 h before inflammasome activation (5 μM nigericin, 150 μg/ml MSU) treated with SFN (10 μM), 15-PGJ2 (10 μM) or DMF (50 μM) (15-PG: 15-PGJ2). Cells and supernatants were harvested after 5 h and analysed for inflammasome activation by ELISA measurement of IL-1p and Western blots as indicated. (C) Freshly isolated PBMCs from human blood were primed overnight with upLPS (100 ng/ml) and treated with SFN (10 μM), tBHQ (10 μM), DMF (50 μM) or 15-PGJ2 (10 μM) 1 h before inflammasome activation by nigericin (5 μM). ELISA measurement for secretion of IL-1β as readout for inflammasome activation was performed after 5 h. (D) HPKs or (E) differentiated and primed THP-1 cells were pretreated with cycloheximide (CHX, 30 μg/ml) to block protein synthesis for 1 h before SFN (10 μM) was added to the cells and after an additional hour the inflammasome was activated by (D) irradiation with UVB or (E) treatment with nigericin (5 μM). Cells and supernatants were harvested after (D) 6 h or (E) 3.5 h and analysed for inflammasome activation by Western blot as indicated. (A-E) Representative experiments performed at least three times are shown. Statistics: (A-C) Error bars represent the mean±SD of a representative experiment performed in triplicates. One-way ANOVA was performed. **P≤0.01; ***P≤0.001.

FIG. 3 shows that Nrf2 activators dampen peritonitis. Mice treated by gavage with (A, B, C) SFN (25 mg/kg) in PBS or (D, E, F) DMF (20 mg/kg) in H₂O containing 0.08% methocel and 10% DMSO, vehicle-treated mice served as control. Regime for (A) SFN or (D) DMF treatment. Peritonitis was induced by peritoneal injection of 2 mg MSU crystals, (B, E) After 6 h the number of neutrophils of the peritoneal lavage was determined by flow cytornetry. (C, F) At the same time, the liver was isolated and analysed for the expression of the Nrf2 target genes Gstp1, Nqo1 and Srxn1 by qRT-PCR. Statistics: Student's t-test. (B, C) n≥3, (E, F) n=7. *P≤0.05; **P≤0.01

FIG. 4 shows that Nrf2 is degraded upon NLRP3 inflammasome activation. (A-C) HPKs were irradiated with (A) UVB or treated with (B) nigericin (5 μM) and cells and supernatants were harvested at different time points as indicated. Western blots for expression and activation of the indicated proteins. Specific bands are marked with an asterisk. (C) Expression of Nrf2 and Nrf2 target genes was determined by qRT-PCR. (D, E) THP-1 cells were differentiated with PMA (27 nM) for 3 d, primed with upLPS (100 ng/ml) overnight and treated with nigericin (5 μM) or MSU (150 μg/ml). Cells and supernatants were harvested at different time points as indicated and analysed for (D) expression and activation of the proteins as indicated by Western blot and (E) expression of Nrf2 and Nrf2 target genes by qRT-PCR. (F) HPKs were transfected with specific siRNAs as indicated (scr: scrambled, c1: caspase-1, K1; Keap1), 2 d later irradiated with UVB or treated with nigericin (5 μM). Cells were harvested after 1 h or 5 h, and lysates were analysed for the expression of the indicated proteins by Western blot. (G, H) Differentiated and primed THP-1 cells with knockout of the indicated genes were treated with (G) nigericin (5 μM) or (H) additionally pretreated with SFN (10 μM). 3.5 h later lysates and supernatants were harvested and analysed for the expression and activation of the indicated proteins by Western blot. (G) Nrf2 expression related to the indicated background band was quantified from the Western blot. A ratio of nigericin-treated to mock-treated samples was calculated. (I) Differentiated and primed THP-1 cells were treated with nigericin (5 μM). After 1 h, cells were harvested directly (1 h) or treated with MG132 (1 μM) or mock-treated. After 2.5 h, cells were harvested and analysed for the expression of the indicated proteins by Western blot. (A-I) Representative experiments performed at least three times are shown. Statistics: (C, E) One-way ANOVA. *P≤0.05; **P≤0.01; ***P≤0.001

FIG. 5 Nrf2 and SFN influence the NLRP3 inflammasome by different mechanisms. (A) HPKs were transfected with scrambled siRNA for control or with Rbx1-specific siRNA, 3 d later cells were harvested and IPs were performed with a caspase-1-specific or with an HA antibody, the latter served as isotype control. Western blots for caspase-1 and Rbx1. A caspase-1 inhibitor was not used. (B) HPKs were transduced with lentiviral constructs encoding FLAG-tagged caspase-1 or GFP under the control of a Tet-On inducible promoter. After selection for 3 d expression was induced by the addition of doxycycline (1 μg/ml). Cells were harvested after 24 h and IP was performed with an ANTI-FLAG® M2 Affinity Gel (Sigma). Western blots showing expression and interactions of the indicated proteins. (C) HPKs were transfected with scrambled or with Rbx1-specific siRNA. After 2 d, the cells were treated with SFN (50 μM) or the solvent DMSO and 1 h later harvested. IPs were performed with a caspase-1-specific antibody, an antibody against HA served as isotype control. Western blots for caspase-1 and Rbx1. (D, E) DCs were differentiated from the bone marrow of wt and Nrf2 knockout mice (n=4), primed overnight with upLPS and treated with the solvent DMSO (ctrl) or SFN (10 μM). After 1 h, BMDCs were treated with 5 μM nigericin and harvested 4.5 h later. (D) Western blots for expression and activation of the indicated proteins and (E) ELISA for quantification of secretion of IL-1p. (F) THP1 cells (3 d differentiated with TPA, overnight primed with LPS) were stimulated with SFN (10 μM) or the solvent DMSO and mock-treated or with nigericin (5 μM) for 2.5 h. Lysates were harvested in Triton buffer and analysed for soluble and insoluble (indicating speck formation) ASC or in DSS-containing buffer for detection of ASC monomers, dimers and oligomers by Western blotting. IL-1β secretion was determined by ELISA. (A-C) Specific bands are marked with an asterisk. (A-E) Representative experiments performed at least three times are shown. (E) Error bars represent the mean±SD of a representative experiment performed in triplicates.

FIG. 6: Bone marrow (BM) cells were isolated from Nrf2-deficient mice and wt litterfnates and differentiated into dendritic cells (DCs). (A, B) After priming with upLPS overnight, BMDCs were treated with the NLRP3 inflammasome activators nigericin (20 μM), zymosan (20 μg/ml), MSU (150 μg/ml), ATP (5 mM) or transfected with poly(dA:dT) (1 μg/ml) for activation of the AIM2 inflammasome. After 6 h, supernatants were analysed for secretion of IL-1β by (A) ELISA or (B) Western blot. Mock-treated but primed BMDCs were analysed for expression of inflammasome proteins and pro-IL-1β at the (C) mRNA level by qRT-PCR or at the (D) protein level by Western blot. (B, D) Duplicates from two individual mice per genotype are shown (biological replicates). (E-G) HPKs were treated with the Nrf2 activators SFN (10 μM), tBHQ (10 μM), DMF (50 μM) and 15d-PGJ2 (10 μM). After 1 h, the cells were harvested and analysed for expression of the indicated proteins using total cell lysates (E) or cytoplasmic and nuclear lysates (F). Western blots for the nuclear protein lamin NC and the cytoplasmic protein α-tubulin served as controls. Specific bands are marked with an asterisk. (G) Total RNA was isolated after 8 h and qRT-PCR was performed for quantification of expression of the Nrf2 target genes glutamate-cysteine ligase, catalytic subunit (GCLC), glutamate-cysteine ligase, modifier subunit (GCLM), and NAD(P)H dehydrogenase, quinone 1 (N001). (A-G) Representative experiments performed at least three times are shown. Statistics: (A) Error bars represent the mean±SD of a representative experiment performed with three mice per genotype. Mann-Whitney test was performed. (G) Error bars represent the mean±SD of a representative experiment. Mann-Whitney test was performed. *P≤0.05

FIG. 7: (A-C) HPKs were transfected with siRNAs for 3 d as indicated. Scrambled (scr) siRNA and siRNA targeting the unrelated vascular endothelial growth factor (VEGF) served as controls. Western blots of (A) total lysates or (B) nuclear and cytoplasmic lysates and (C) qRT-PCR for expression of target gene expression. (D) HPKs were treated with MG132 (1 μM), PDTC (500 μM) or BAPTA-AM (12.5 μM) for 10 min and harvested (before UV) or irradiated with UVB and harvested after 1 h. Western blots showing expression of Nrf2. (E) Differentiated and primed THP-1 cells were pretreated with cycloheximide (CHX, 30 μg/ml) for 1 h before priming cells with upLPS overnight. Western blots show expression of the indicated proteins. (A-E) Representative experiments performed at least three times are shown. Statistics: (C) Mann-Whitney test, related to scr control. *P≤0.05

FIG. 8: (A-C) HPKs were treated with the Nrf2 activating compounds (A) SFN, (B) DMF, and (C) 15d-PGJ2, irradiated with UVB 30 min later and harvested after 5 h. Analogous experiment as described in FIG. 2A for tBHQ. (D) THP-1 cells were differentiated with TPA for 3 d and primed with LPS overnight. Then, cells were treated with the solvent DMSO, SFN (10 μM) or 15-PGJ2 (10 μM). After 30 min the inflammasome was activated by nigericin treatment and cells and supernatants were harvested after 2.5 h. Western blots showing prolL-1β and mature IL-1p in the lysate and supernatant. 20% lysate and supernatant of a 12 well was used, respectively, in order to allow a comparison. IL-1β was quantified in the supernatant by ELISA and cytotoxicity by LDH release.

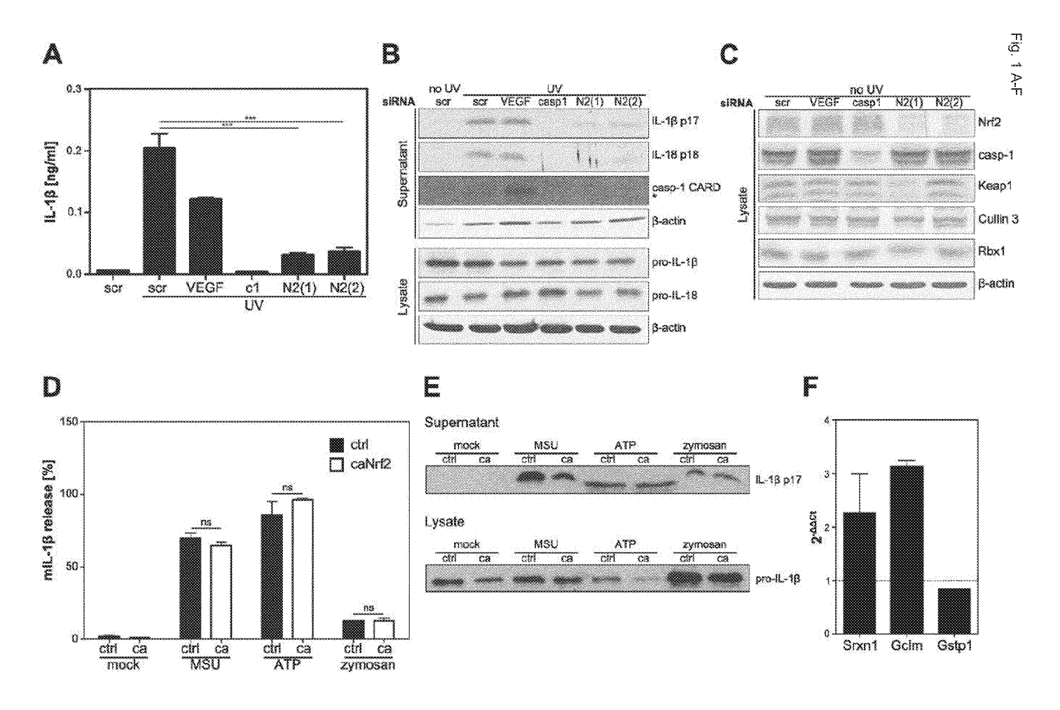

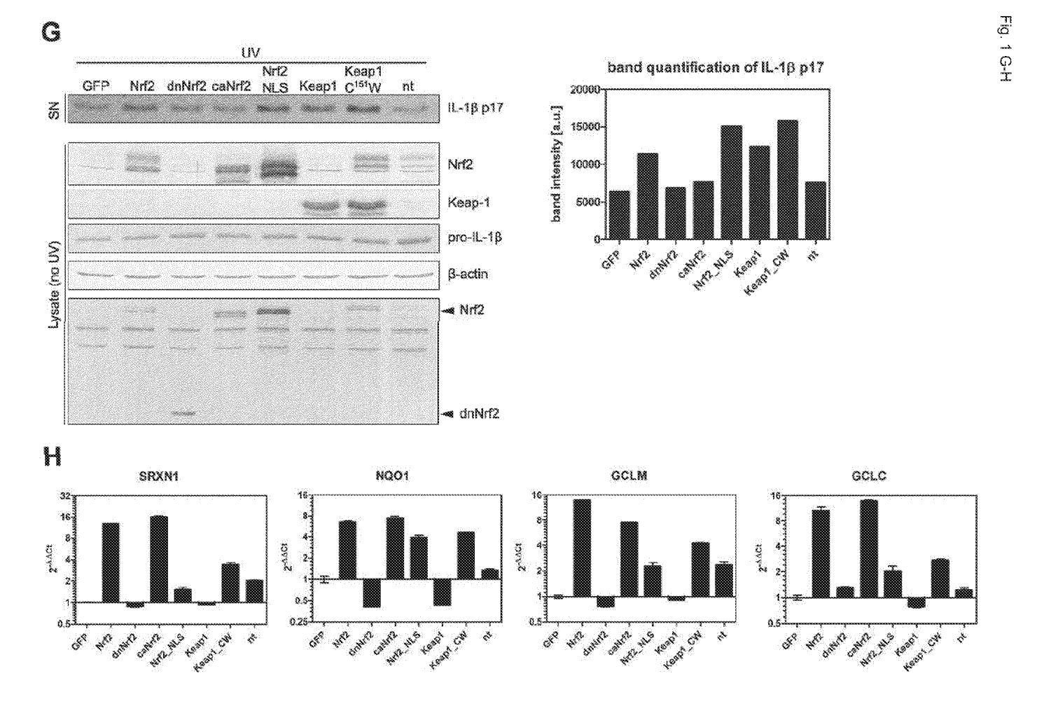

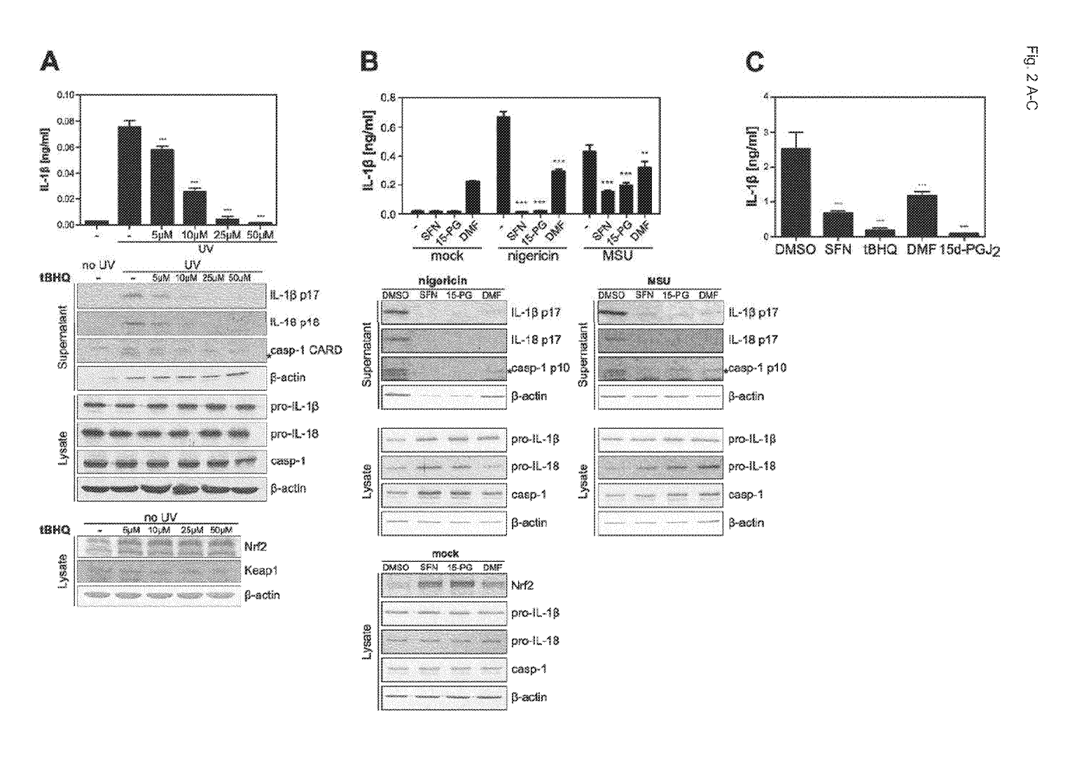

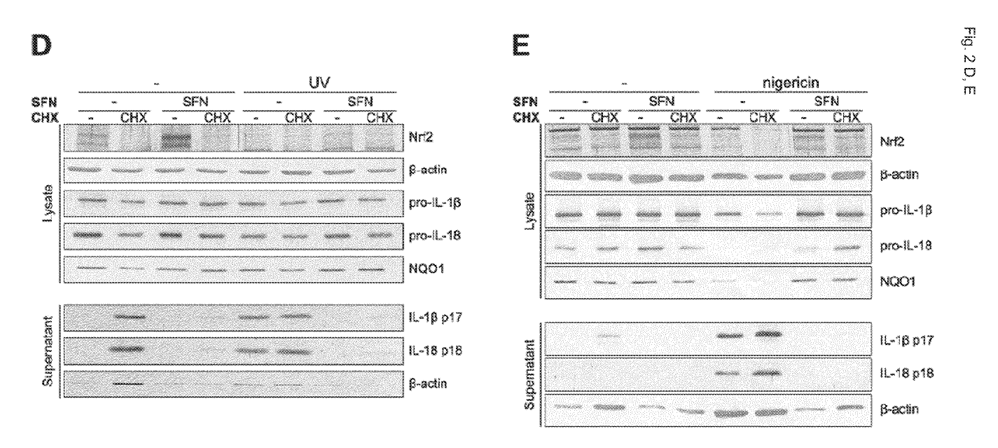

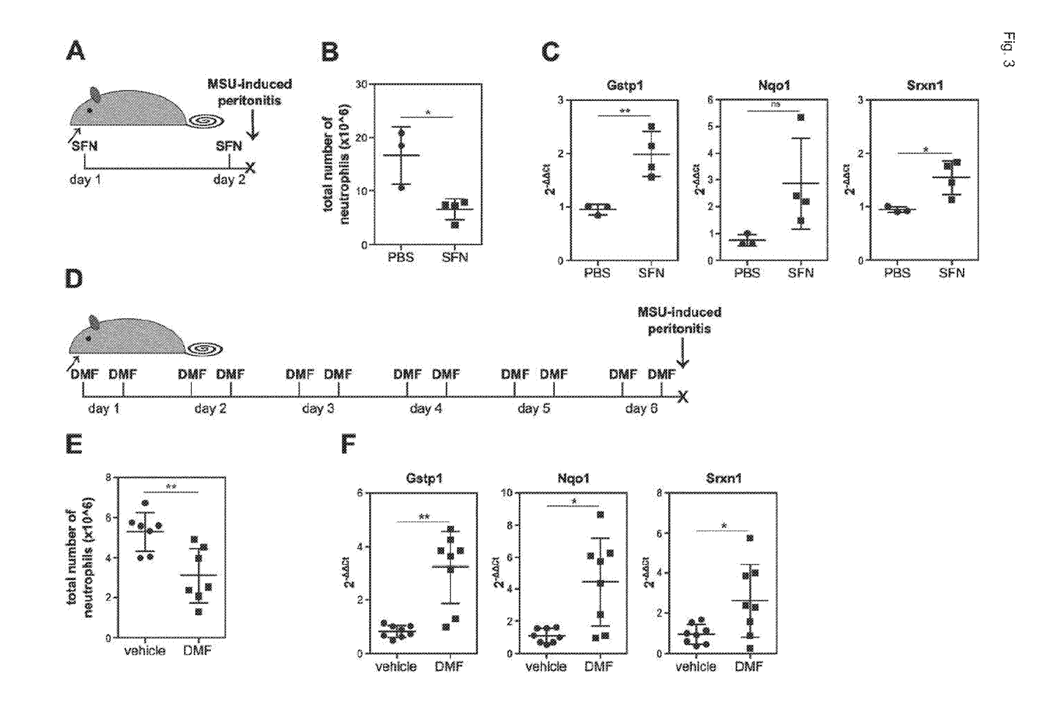

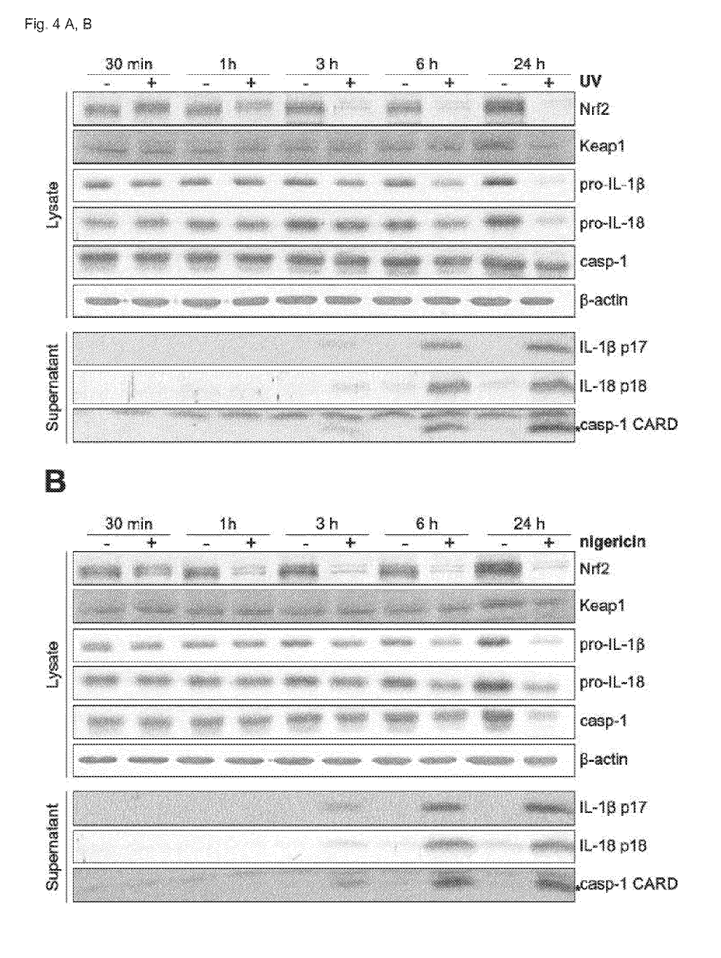

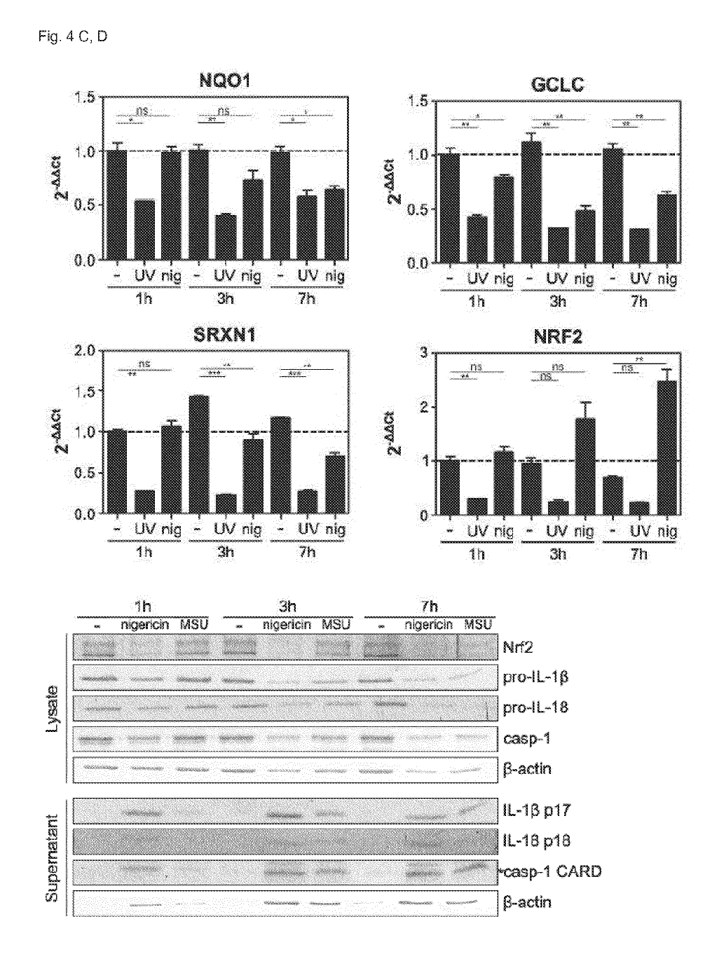

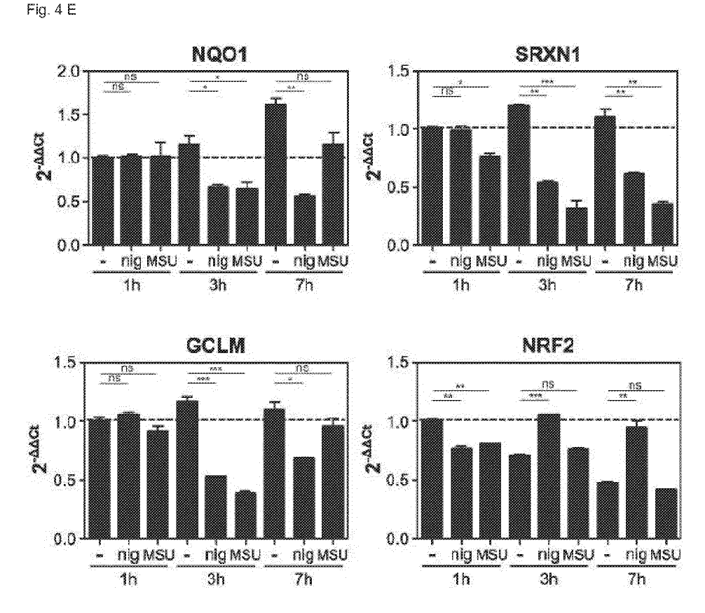

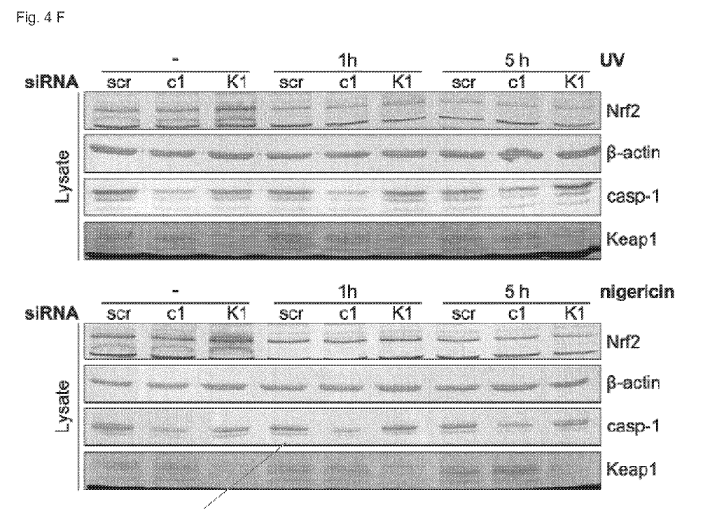

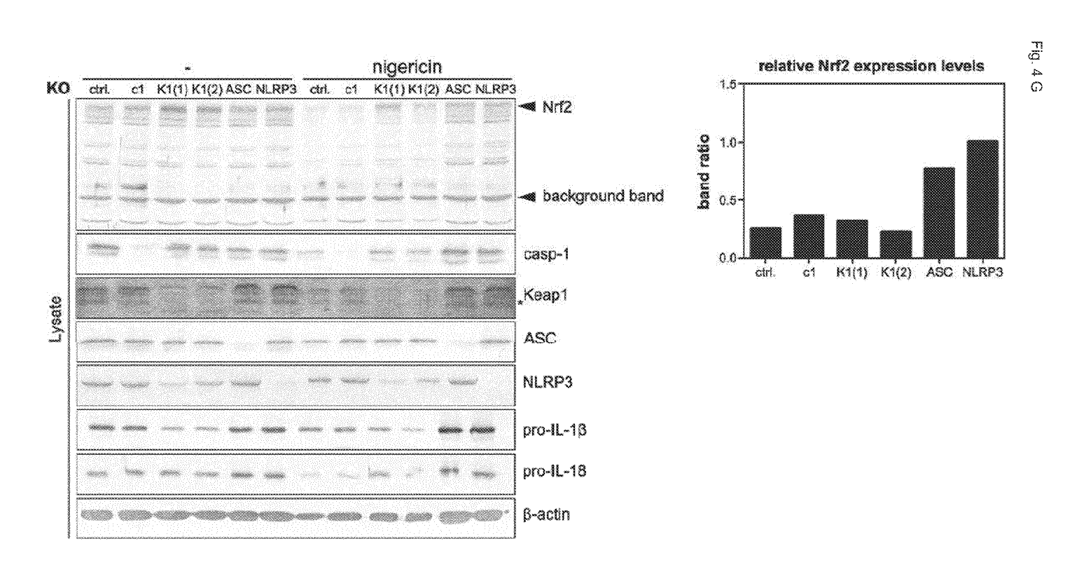

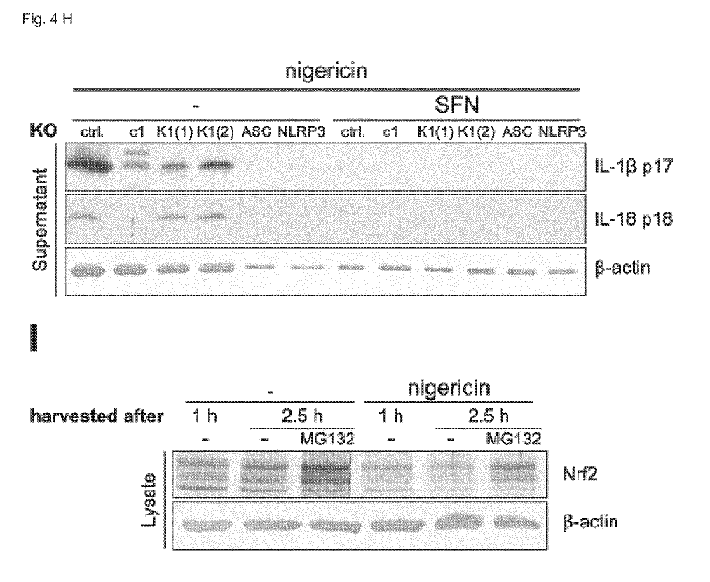

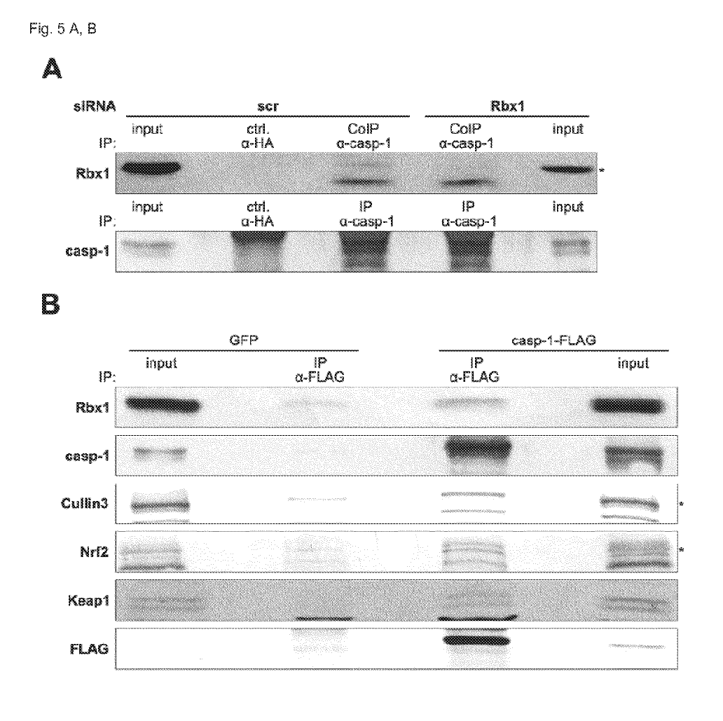

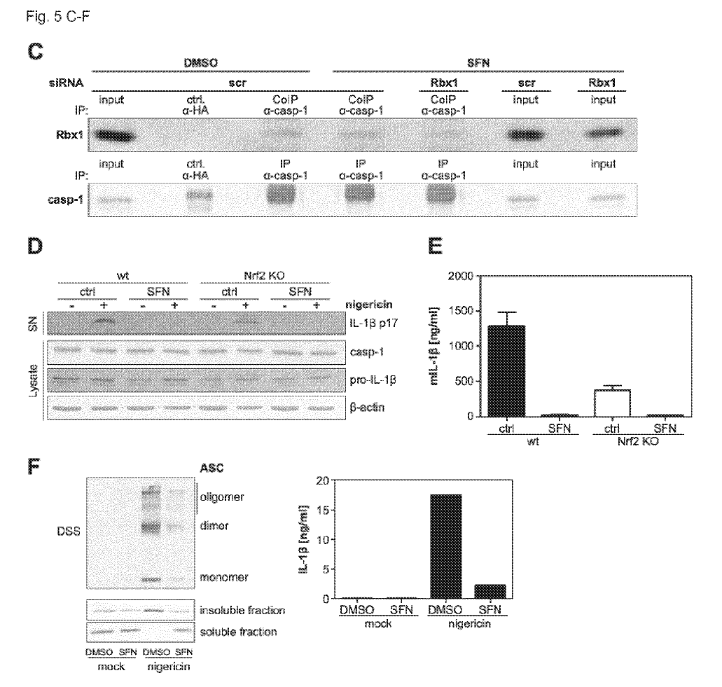

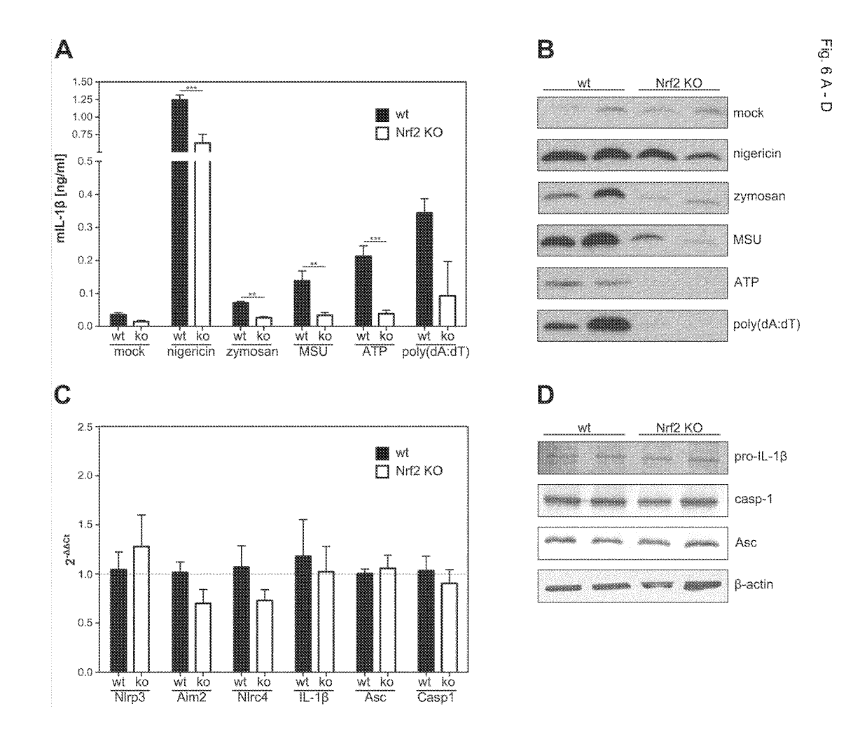

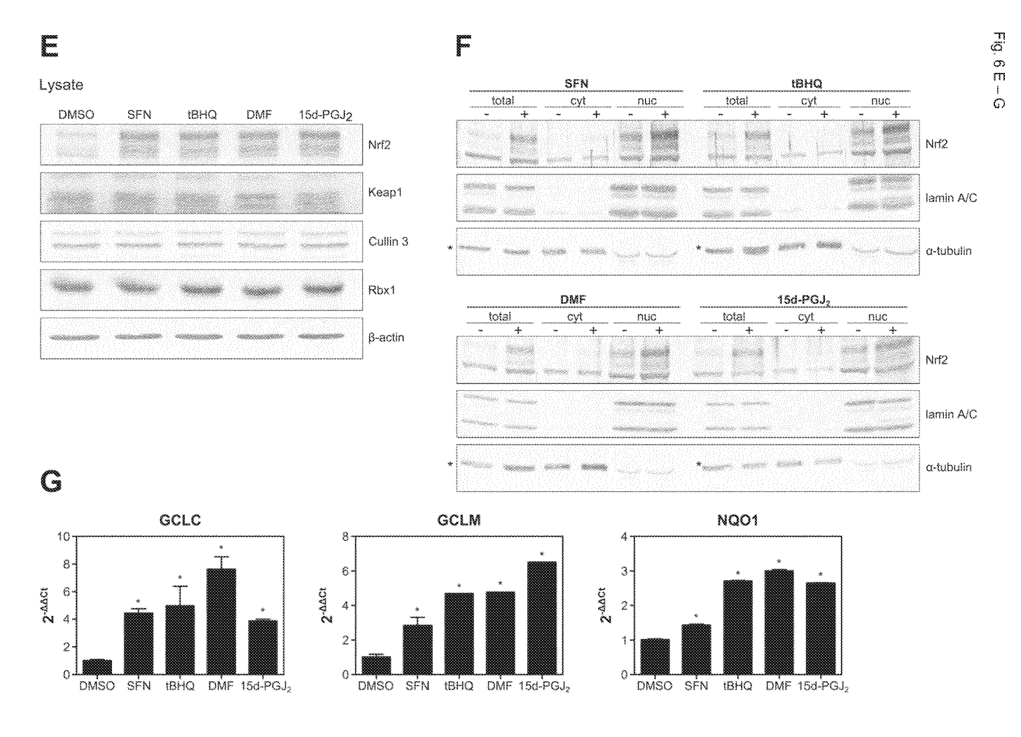

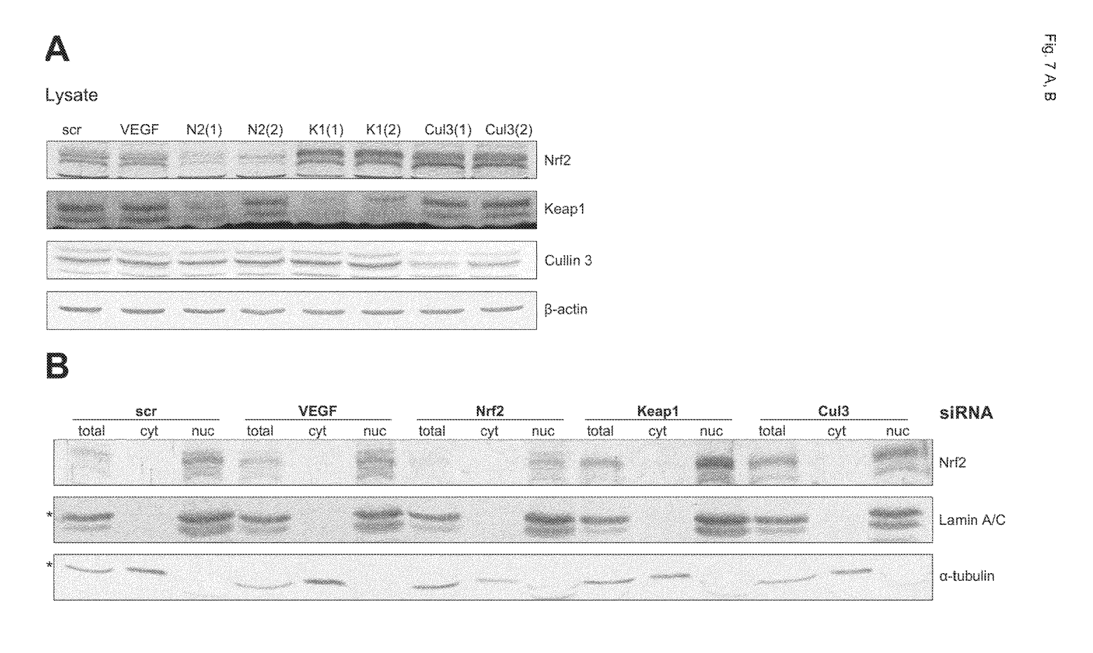

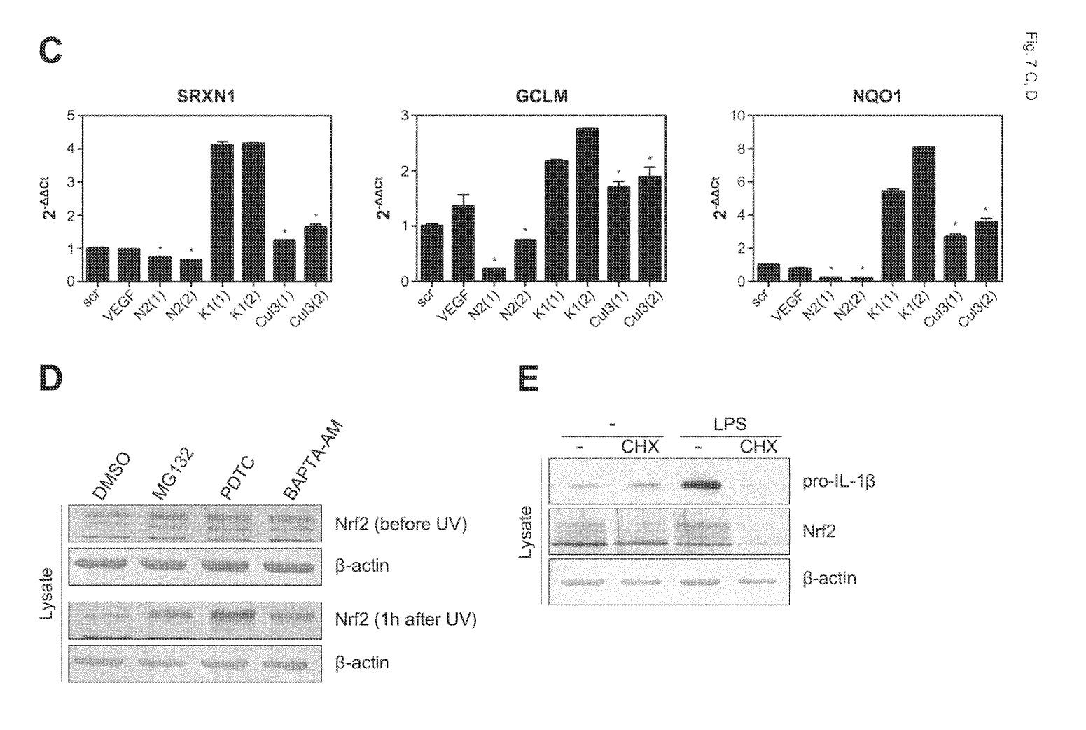

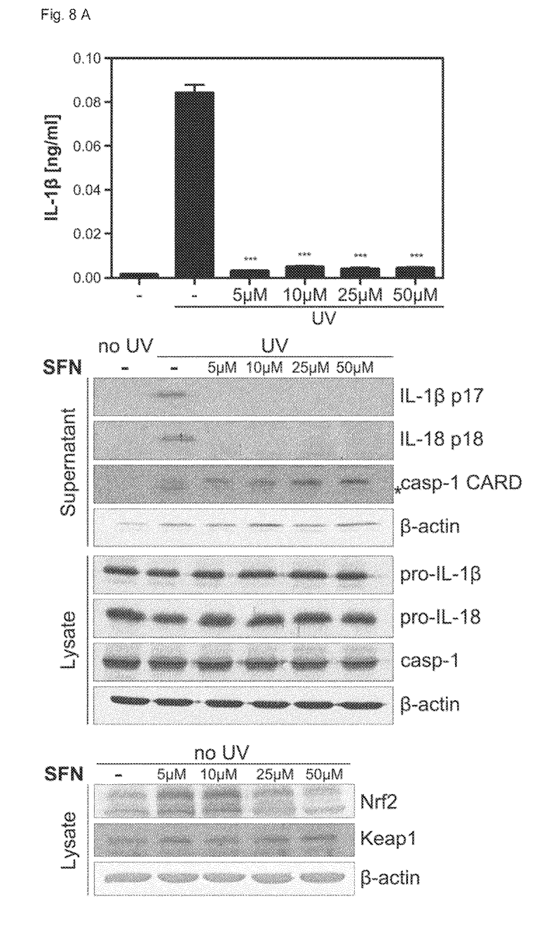

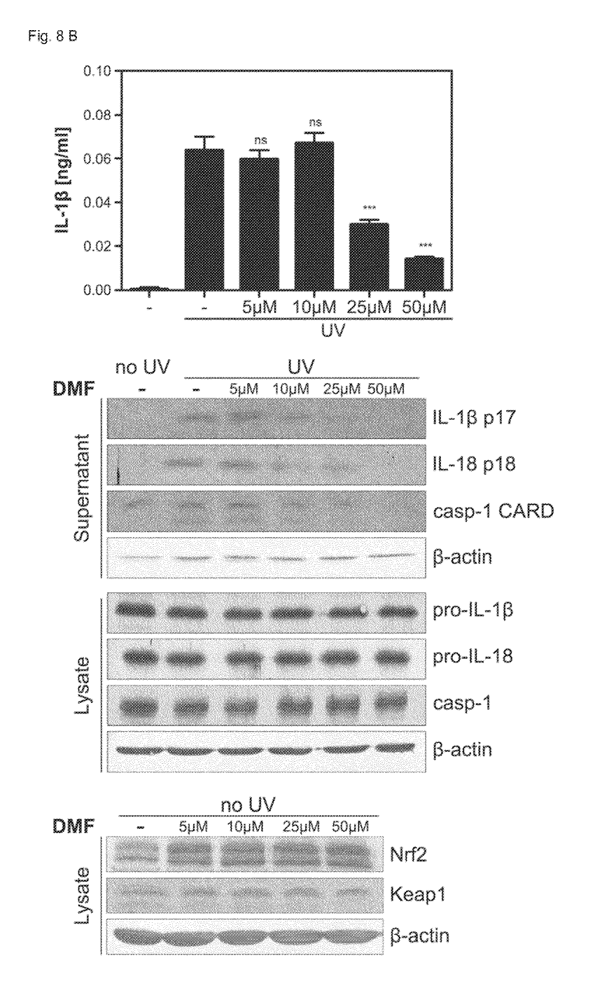

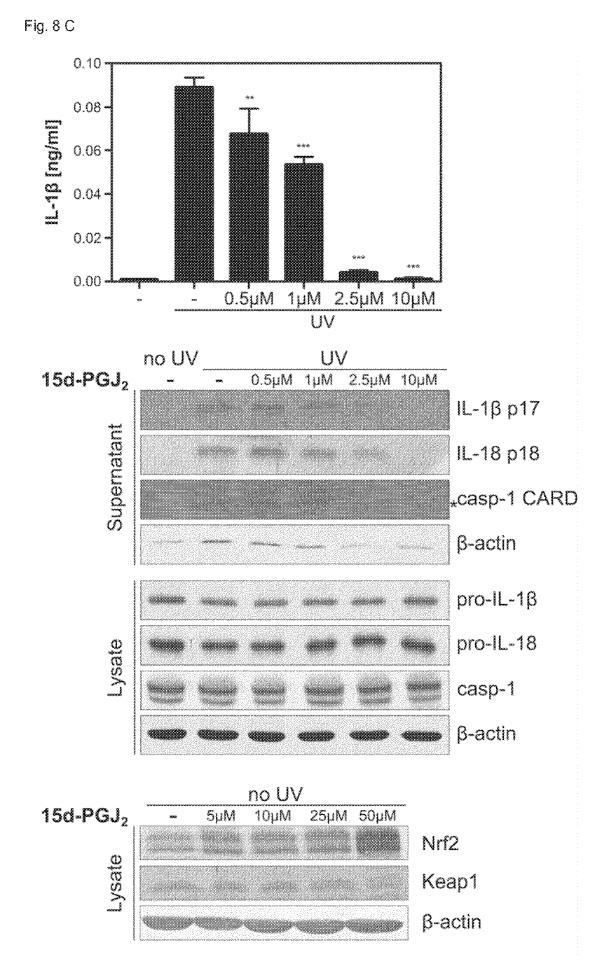

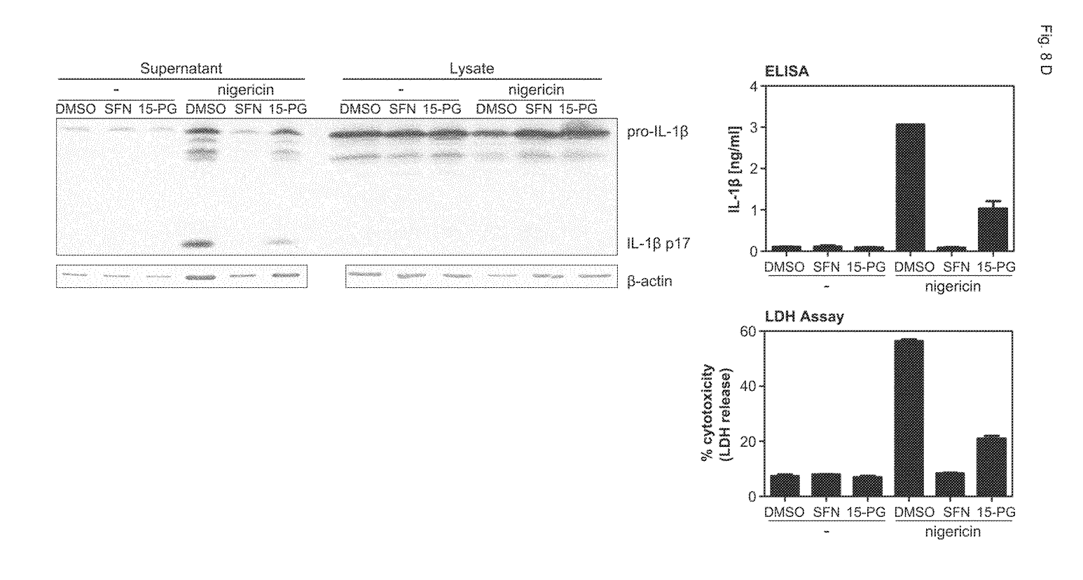

1. A compound specified by formula (I) 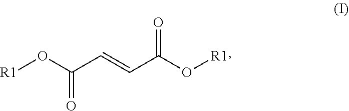
wherein each R1 is independently selected from C₁-C₆ alkyl, for use in prevention or therapy of gout, acne, type 2 diabetes, Vitiligo and/or pyoderma gangrenosum.

2. The compound specified by formula (I), wherein each R1 is methyl (CH₃), for use in prevention or therapy of gout, acne, type 2 diabetes, Vitiligo and/or pyoderma gangrenosum.

3. A dosage form comprising the compound according to claim 1 for use in prevention or therapy of gout, acne, type 2 diabetes, Vitiligo and/or pyoderma gangreno sum.

4. A method of treatment or prevention of gout, acne, type 2 diabetes, Vitiligo and/or pyoderma gangrenosum, comprising administration of the compound according to claim 1 to a patient in need thereof.

5. A dosage form comprising the compound according to claim 2 for use in prevention or therapy of gout, acne, type 2 diabetes, Vitiligo and/or pyoderma gangrenosum.

6. A method of treatment or prevention of gout, acne, type 2 diabetes, Vitiligo and/or pyoderma gangrenosum, comprising administration of the compound according to claim 2 to a patient in need thereof.
